(19) United States
(12) Plant Patent Application Publication
(10) Pub. No.: US 2003/0208807 P1
(43) Pub. Date: Nov. 6, 2003

(54) Prunus L. variety named 'Piku 1'

(76) Inventor: Brigitte Wolfram, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 10/136,465

(22) Filed: May 1, 2002

(51) Int. Cl. A01H 5/00

(52) U.S. Cl. PLT/183

(57) ABSTRACT

A new and distinct Prunus rootstock compatible with other Prunus varieties and producing trees with reduced growth, hardiness to frost and high productivity.

### CLASSIFICATION

Prunus L.

### VARIETAL DENOMINATION

‘Piku 1’

### BACKGROUND OF THE INVENTION

The present invention comprises a new and distinct cultivar of hybrid Prunus known by the varietal name ‘Piku 1’. The new variety was discovered in 1972 in a selective breeding program designed to produce growth reducing rootstock for cherry trees in Dresden-Pilluitz, Germany as a cross between an unnamed, unpatented variety of Prunus avium (female parent) and an unnamed, unpatented variety of Prunus tomentosa (male parent). The new variety differs from its parents in its growth habit. The following characteristic distinguish the new variety from Prunus avium varieties:

1. Three-way hybrid.

2. Reduces growth in grafted varieties by 30%.

3. Precocious, about 1-3 years.

4. Higher productivity.

5. Propagated by green cuttings or tissue culture.

6. High resistance to winter frost.

7. Leaf margins roll inwards.

The new variety was first asexually reproduced via green cuttings and tissue culture in 1975 in Dresden-Pilluitz, Germany and subsequent propagation therein has confirmed that the characteristics of the new variety are stable and are transmitted to subsequent generations.

### DESCRIPTION OF THE DRAWINGS

The accompanying photographic drawings illustrate the new variety, with the color being as nearly true as is possible with color illustrations of this type.

FIG. 1 shows the tree of the new variety;

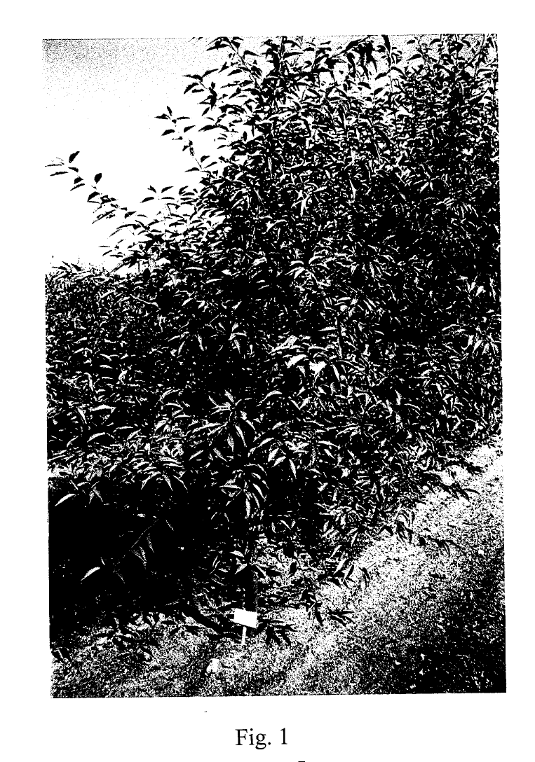

FIG. 2 shows a branch with leaves;

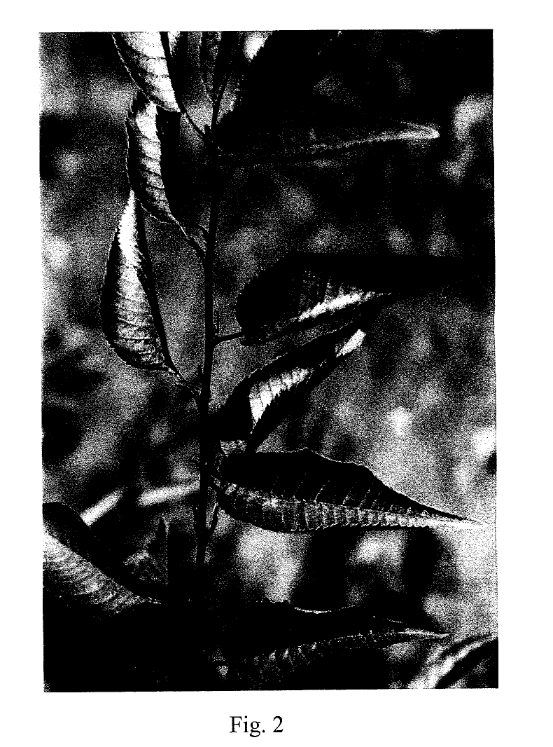

FIG. 3 shows a branch with flower buds; and

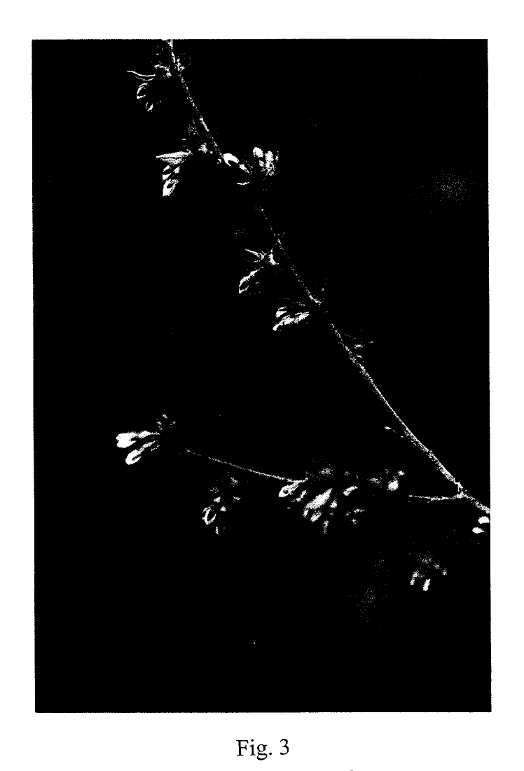

FIG. 4 shows a branch with opened flowers.

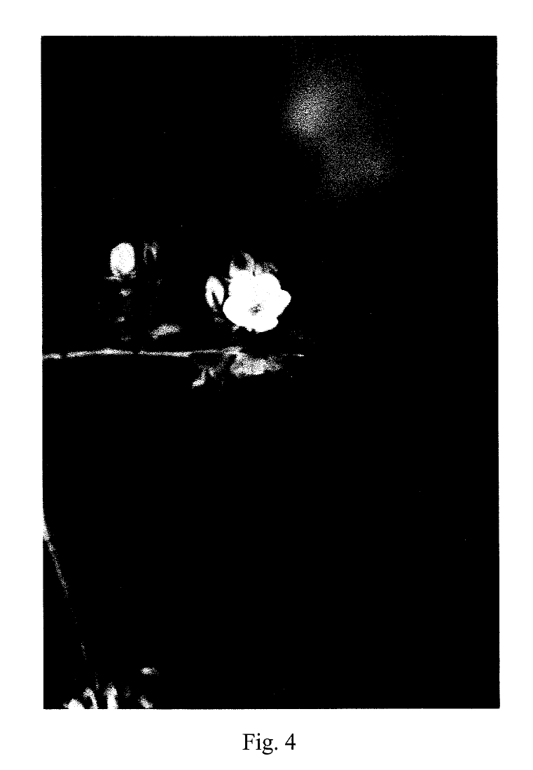

### DESCRIPTION OF THE PLANT

The following detailed description sets forth the characteristics of the new cultivar. The data which defines these characteristics were collected by asexual reproductions via green cuttings and tissue culture carried out in Dresden-Pilluitz, Germany. The color measurements were taken in outdoors Dresden-Pilluitz, Germany. Color designations are noted with reference to The Royal Horticultural Society Colour Chart. The new variety prefers dry soil and is resistant to PNRV and PDV. As a rootstock, it can be reproduced via softwood cuttings. The new variety anchors well with few root sprouts, good compatability with grafts and exhibits good growth vigor.

### TREE

Size: Medium.

Form: Upright, broad.

Trunk:

Size.—Medium.

Surface texture.—Exfoliating.

Bark color.—Middle brown to dark brown.

Branches:

Surface texture.—Smooth.

Bark color.—Middle brown.

New growth color.—Light-dark red.

Mature growth color.—Dark brown.

Internode length.—About 1.8 cm.

Lenticels.—Small.

Bud.—Small.

Leaves:

Length.—9.1 cm.

Width.—4.0 cm.

Surface texture.—Fine, smooth.

Form.—Elongated, elliptical.

Color.—Light green.

Margin.—Rolls towards upper surface.

Petioles:

Length.—1.4 cm.

Thickness.—Thin.

Leaf glands:

Quantity.—1-2.

Position.—On the end of the leaves.

Size.—Small.

Color.—Red-brown.

Stipules:

Quantity.—Rare.

Size.—Small.

Flowers:

Size.—Medium.

Color.—White.

Number.—Many.

Sexual characteristics.—Diploid.

### FRUIT

Maturity date/location: Early in Dresden, Pilluitz.

Size: Small; low crop yield.

Axial Diameter: 1-1.5 cm.

Skin: Thin, soft, dark

I claim:

1. A new and distinct variety of Prunus rootstock named ‘Piku 1’ substantially as shown and described.
